(19) United States
(12) Patent Application Publication
(10) Pub. No.: US 2022/0273022 A1
(43) Pub. Date: Sep. 1, 2022

(54) COMBUSTIBLE TOBACCO PRODUCT DESIGN SYSTEM AND METHOD

(71) Applicant: British American Tobacco (Investments) Limited, London (GB)

(72) Inventors: Jailson Cardoso DIAS, London (GB); Samuel KAISER, London (GB); Marcelo Caetano Alexandre MARCELO, London (GB); Frederico L. F. SOARES, London (GB); Jorge A. A. QUINTERO, London (GB); Daniel D. CONTY, London (GB); Oscar Francisco Swenson PONTES, London (GB); Guilherme Post SABIN, London (GB)

(21) Appl. No.: 17/630,342

(22) PCT Filed: Jul. 24, 2020
(86) PCT No.: PCT/GB2020/051796

§ 371 Date: Jan. 26, 2022

(30) Foreign Application Priority Data
Jul. 26, 2019 (GB) 1910738.2

(51) Int. Cl. A24D 1/00

(52) U.S. Cl. CPC A24D 1/002; A24D 1/004; A24D 3/043

(57) ABSTRACT

A method of designing a target combustible tobacco product, the method comprising receiving respective values for a plurality of input parameters (101); calculating respective values for a plurality of design parameters (130) for the combustible tobacco product based on the received values for the plurality of input parameters, and providing the calculated values as an output. The plurality of design parameters comprise at least two parameters selected from: a tobacco blend composition; tar, nicotine and carbon monoxide deliveries; a smoke sensory attribute; a number of puffs associated with the combustible tobacco product; combustible tobacco product dimensions; tobacco weight; tobacco rod and/or filter density; tobacco rod and/or filter firmness; open and/or closed combustible tobacco product pressure drop; filter pressure drop; cigarette paper porosity; and ventilation level.

### PRIORITY CLAIM

The present application is a National Phase entry of PCT Application No. PCT/GB2020/051796, filed Jul. 24, 2020, which claims priority from Great Britain Application No. 1910738.2, filed Jul. 26, 2019, each of which is hereby fully incorporated herein by reference.

### TECHNICAL FIELD

The present disclosure relates to combustible tobacco products, and in particular to systems and methods for designing and simulating combustible tobacco products.

### BACKGROUND

Designing a combustible tobacco product involves the selection of various properties of the combustible tobacco product. For example, designing a filtered cigarette may include selecting tobacco blend; cigarette dimensions; filter type; filter properties; tobacco weight; cigarette density; cigarette firmness; and cigarette paper porosity. The selection of these properties may affect the sensory attributes and the tar, nicotine and carbon monoxide deliveries of the combustible tobacco product.

### SUMMARY

In accordance with a first aspect, this specification describes a method of designing a target combustible tobacco product. The method includes receiving respective values for a plurality of input parameters; calculating respective values for a plurality of design parameters for the combustible tobacco product based on the received values for the plurality of input parameters; and providing the calculated values as an output. The plurality of design parameters includes at least two parameters selected from: a tobacco blend composition; tar, nicotine and carbon monoxide deliveries; a smoke sensory attribute; a number of puffs associated with the combustible tobacco product; combustible tobacco product dimensions; tobacco weight; tobacco rod and/or filter density; tobacco rod and/or filter firmness; open and/or closed combustible tobacco product pressure drop; filter pressure drop; cigarette paper porosity; and ventilation level.

In accordance with a second aspect, the specification describes a computer program including instructions which, when the program is executed by a computer, cause the computer to carry out the method in accordance with the first aspect above.

In accordance with a third aspect, the specification describes a computer-readable storage medium comprising instructions which, when executed by a computer, cause the computer to carry out the method in accordance with the first aspect above.

In accordance with a fourth aspect, the specification describes a data processing apparatus comprising a processor and a computer-readable storage medium in accordance with the third aspect.

In accordance with a fifth aspect, the specification describes a system including a data processing apparatus in accordance with the fourth aspect and a combustible tobacco product manufacturing apparatus. The system is configured to carry out the method in accordance with the first aspect above.

### BRIEF DESCRIPTION OF THE DRAWINGS

Embodiments of the present disclosure will now be described, by way of example only, with reference to the accompanying drawings, in which:

FIG. 1 is a schematic block diagram illustrating a system for designing a combustible tobacco product, according to an embodiment;

FIG. 2 is a schematic block diagram illustrating a system component for calculating design parameters for a combustible tobacco product, according to an embodiment;

FIG. 3 is a flow diagram of a method for designing a combustible tobacco product, according to an embodiment;

FIG. 4 is a flow diagram of a method for performing an optimization procedure directed to deriving a descriptor for a target combustible tobacco product, according to an embodiment;

FIG. 5 illustrates performing an example crossover operation to derive a new combustible tobacco product descriptor based on existing combustible tobacco product descriptors, according to an embodiment;

FIG. 6 is a schematic illustration of a filtered combustible tobacco product, according to an embodiment; and

FIG. 7 illustrates comparisons of estimates of smoke sensory attributes of a combustible tobacco product derived according to example embodiments with sensory attribute values obtained using other methods.

DETAILED DESCRIPTION OF THE DRAWINGS

Example implementations provide system(s) and method(s) for designing and simulating combustible tobacco products. The described systems and methods may facilitate designing and prototyping combustible tobacco products in silico reducing the time and cost of developing new combustible tobacco products. Implementations may also facilitate the design of combustible tobacco products having similar sensory attributes to an existing combustible tobacco product while using a different tobacco blend composition; having different tar, nicotine and/or carbon monoxide deliveries; and/or being in a different format, e.g. a short cigarette having similar sensory attributes to an existing king-size cigarette.

As used herein, the term “combustible tobacco product” includes smokeable products such as cigarettes, cigars and cigarillos whether based on tobacco, tobacco derivatives, expanded tobacco, reconstituted tobacco or tobacco substitutes. Such combustible tobacco products may be provided with a filter for the gaseous flow drawn by the smoker.

Combustible Tobacco Product Design System

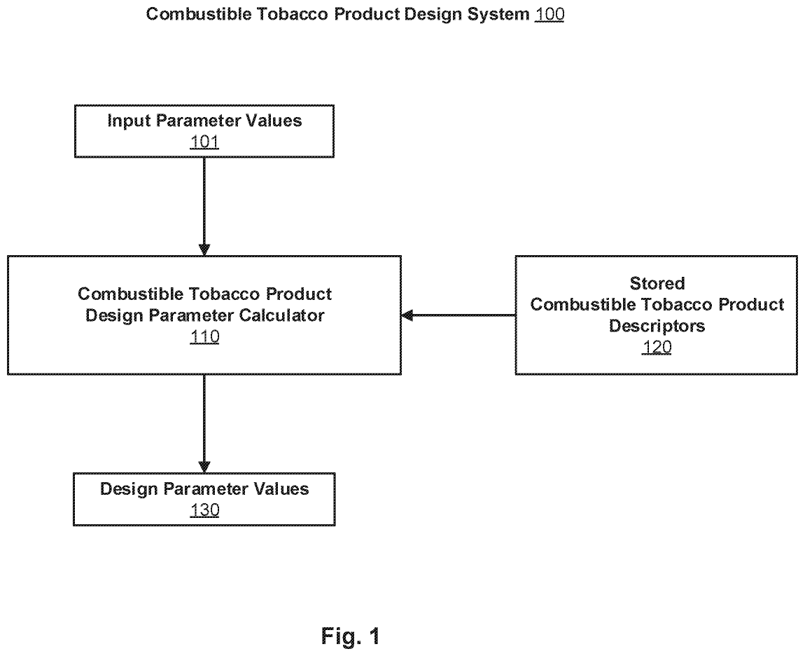

FIG. 1 is a schematic block diagram illustrating a system 100 for designing a combustible tobacco product.

The combustible tobacco product design system 100 is implemented using one or more suitable computing devices. For example, the one or more computing devices may be any of or any combination of one or more desktop computers, one or more notebook computers, one or more tablet computers, one or more workstation computers, one or more mainframe computers, and one or more blade server computers. In embodiments where the combustible tobacco product design system 100 is implemented using a plurality of computing devices, the computing devices may be configured to communicate with each other. The communication may be via one or more peripheral interfaces and/or over one or more networks. The one or more networks may be any of or any combination of the internet, local area networks, cellular networks and wireless networks. The combustible tobacco product design system may be implemented using a numerical computing environment and/or framework, e.g. MATLAB, Mathematica, NumPy and/or R. The combustible tobacco product design system may also be implemented using one or more suitable programming languages. Examples of suitable programming languages are Python, C, C++, C# and Java.

The combustible tobacco product design system 100 includes input parameter values 101, a combustible tobacco product design parameter calculator 110, stored combustible tobacco product descriptors 120 and design parameter values 130.

The input parameter values 101 are desired and/or set values for parameters of a target combustible tobacco product. The parameters may include, but are not limited to, one or more of tobacco blend parameters; smoke sensory attributes; tar, nicotine and/or carbon monoxide deliveries; and parameters describing the physical properties and/or composition of a combustible tobacco product.

Examples of tobacco blend parameters include the proportions of each of a number of tobacco varieties and/or qualities. Examples of macro tobacco variety groups include flue-cured Virginia, air-cured Burley, specially processed, sun-cured Oriental, Cavendish style, stem, reconstituted tobacco and tobacco or reconstituted tobacco formed from non-stem tobacco by-products. Varieties of flue-cured Virginia tobacco include Lemon, Orange and Mahogany 1 tobacco varieties. Varieties of air-cured Burley tobacco include Light Mahogany, Mahogany 2 and Dark Mahogany. Varieties of specially processed tobacco include Fermented Dark Air-Cure, Dark Fire-Cured, and Galpão Comum. Varieties of sun-cured Oriental tobacco include Samsun, Basma, and Izmir. Reconstituted tobacco, for instance formed from tobacco by-products and/or stem, includes the tobacco material as described in PCT Pub. No. WO2006061117 and U.S. Pat. No. 5,562,108, the contents of each of which are incorporated herein by reference. At least some of these tobacco varieties are available in several quality grades, e.g., high quality and medium quality.

The tobacco blend parameters may include indications that tobacco of one or more given variety groups, varieties and/or qualities should or should not be included in the target combustible tobacco product. For example, the parameters may indicate that a tobacco blend of flue-cured Virginia tobacco, tobacco stem and reconstituted tobacco is desired.

Examples of smoke sensory attributes include draw effort, mouthful of smoke, impact, irritation, mouth drying, mouth coating, taste intensity, tobacco aroma, brightness and/or darkness. The smoke sensory attributes may be represented using numerical values which are indicative of the sensory impression of a combustible tobacco product on consumers according to data and/or models derived using consumer surveys and/or focus groups.

Examples of parameters describing the physical properties and/or composition of the combustible tobacco product include a number of puffs associated with the combustible tobacco product, for instance the maximum number of puffs achievable from the product under a standard smoking regime, cigarette dimensions including at least one of tobacco column length, filter plug length, tipping length and circumference of product, net tobacco weight, filter plug pressure drop (for instance encapsulated pressure drop), total cigarette pressure drop, for instance with any ventilation openings open and/or closed, ventilation rate, firmness and cigarette density. The firmness and/or density may, for instance, be the firmness or density of the tobacco rod or of the filter. Firmness can, for instance, be measured using hardness measurement equipment supplied by Borgwaldt or others and based on product diameter measurements before and after the product has been subjected to a given load. The density can be calculated as the weight of a component of the product per unit of volume for that component.

The design parameter values 130 are calculated values for a number of design parameters of the target combustible tobacco product. The design parameters may be any number of the parameters described above in relation to the input parameters 101. The design parameters may include one or more parameters of the combustible tobacco product which were not input parameters.

The design parameters may be understood as parameters for which values are to be chosen such that the target combustible tobacco product has the provided values for the input parameters, or as close as is achievable. For example, the input parameter values may indicate that the target combustible tobacco product is desired to have certain sensory attribute values and have a blend consisting of given tobacco varieties; and the values for the design parameters may describe the physical properties and/or composition of the target combustible tobacco product and the proportions of the tobacco varieties in the blend such that the target combustible tobacco product has properties matching, or at least resembling, the received values for the input parameters.

The combustible tobacco product design parameter calculator 110 receives the input parameter values 101, and calculates the design parameter values 130 for a combustible tobacco product based on the received input parameter values 101.

In calculating the design parameter values 130, the combustible tobacco product design parameter calculator 110 may derive a target combustible tobacco product descriptor. Combustible tobacco product descriptors may include values for the design parameters and values for the input parameters. The values of a given combustible tobacco product descriptor for the design parameters and input parameters may be unscaled values for the parameters, i.e. each of the values may be of the same scale as the corresponding input parameter or design parameter value. Alternatively, the values of a given combustible tobacco product descriptor for the design parameters and input parameters may have undergone feature scaling, e.g. each the values for the parameter may have been rescaled using an appropriate method such as min-max normalization, mean normalization or standardization. Different rescaling methods may be appropriate for different parameters and, as such, the values of a given combustible tobacco product descriptor for different parameters may be rescaled according to different methods. In some instances, the values of a given combustible tobacco product descriptor for some of the parameters may have undergone feature scaling while others may have not. Where the values of the target combustible tobacco product descriptor have undergone feature scaling, the combustible tobacco product design parameter calculator 110 may transform at least the values of the target combustible tobacco product descriptor into an appropriate scale for the design parameter values, e.g. design parameter values understandable by a design system user and/or usable for manufacturing the target combustible tobacco product.

Combustible tobacco product descriptors may be implemented using any suitable data structure. Suitable data structures include, but are not limited to, arrays, vectors, matrices, rows and/or columns of matrices, in-memory objects, markup language files, serialized binary data, database entries and text data.

The target combustible tobacco product design parameter calculator 110 may derive the target combustible tobacco product descriptor by performing an optimization procedure, which may be a stochastic optimization procedure. For example, the optimization procedure may be any of particle swarm optimization, ant colony optimization, simulated annealing, a Monte Carlo algorithm, Runge-Kutte methods, a genetic algorithm, or any combination thereof. Where a genetic algorithm is used, it may be a real coded genetic algorithm. The optimization procedure may be directed towards deriving a target combustible tobacco product having a maximal fitness. The fitness of a given combustible tobacco product descriptor may be based on differences between the input parameter values 101, or a feature scaling thereof, and the corresponding values of the target combustible tobacco product descriptor.

The fitness of a given combustible tobacco product descriptor may be measured using a fitness function or loss function. Where a fitness function is used, a greater value of the fitness function for the given combustible tobacco product descriptor indicates a greater fitness. Where a loss function is used, a lesser value of the loss function for the combustible tobacco product descriptor indicates a greater fitness. For example, the fitness of a combustible tobacco product descriptor may be inversely related to the root mean square deviation, also referred to as the root mean square error, between the input parameter values 101, or a feature scaling thereof, and the corresponding values of the target combustible tobacco product descriptor, and, this root mean square deviation used as a loss function. This root mean square deviation may be denoted as:

$$\frac{1}{N} \sqrt{{\sum }_{i = 1}^{N} {( {p}_{i} - {c}_{i} )}^{2}} ,$$

where N is the number of input parameters, pᵢ is the ith input parameter value, or a feature scaling thereof, and cᵢ is the value of a given combustible tobacco product descriptor for the ith input parameter.

The stored combustible tobacco product descriptors 120 may be used by the combustible tobacco product design parameter calculator 110 in the derivation of the design parameter values 130. For example, the stored combustible tobacco product descriptors may be used to derive the target combustible tobacco product descriptor. The stored combustible tobacco product descriptors 120 may be implemented using any suitable data structure for combustible tobacco product descriptors, including those previously referred to. The stored combustible tobacco product descriptors 120 may be stored using any suitable data storage mechanism, e.g. file system storage, database storage or an in-memory cache. The stored combustible tobacco product descriptors 120 may have been derived using measurements of physical qualities and properties; chemometric analysis; and/or results of consumer focus groups and/or panels. Some of the stored combustible tobacco product descriptors 120 may have been derived using a chemosensory model such as that described in PCT Pub. No. WO2018007789, the contents of which are incorporated herein by reference.

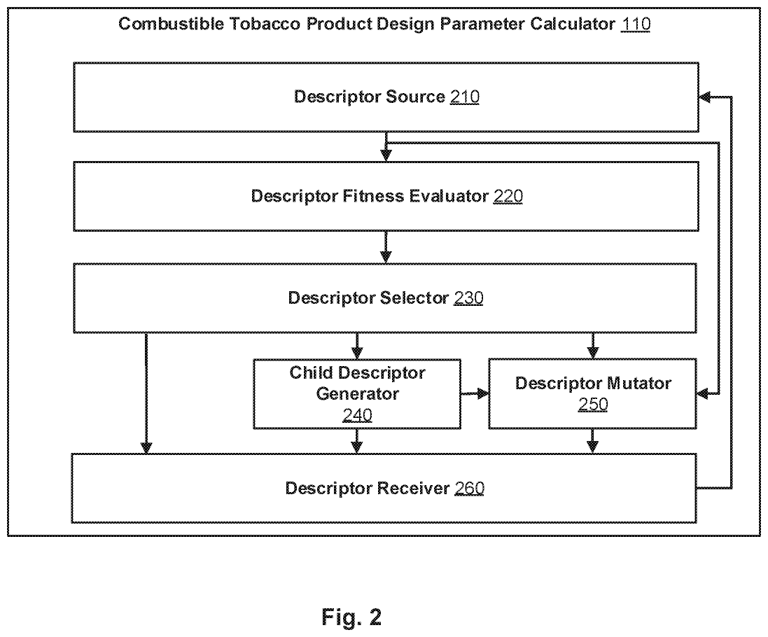

The target combustible tobacco product descriptor may be derived by using a plurality of the stored combustible tobacco product descriptors, or a feature scaling thereof, as initial combustible tobacco product descriptors. The combustible tobacco product design calculator 110 may evaluate the fitness of the initial combustible tobacco product descriptors and derive new combustible tobacco product descriptors based on a selected subset of them, e.g. the fittest J initial combustible tobacco product descriptors may be used to derive the new combustible tobacco product descriptors. The fitness of these new combustible tobacco product descriptors may then be evaluated and a selected subset of the new combustible tobacco product descriptors used to generate a further generation of combustible tobacco product descriptors. Subsequent generations may then be generated, each of the subsequent generations derived from a selected subset of the combustible tobacco product descriptors of the preceding generation. The target combustible tobacco product descriptor may be the fittest combustible tobacco product descriptor of the last generation. A related example embodiment of the combustible tobacco product design parameter calculator 110 is described in relation to FIG. 2.

The combustible tobacco product design system 100 may also include a combustible tobacco product manufacturing apparatus (not shown). The design parameter values may be provided to the combustible tobacco product manufacturing apparatus and used to manufacture the target combustible tobacco product.

Combustible Tobacco Product Design Parameter Calculator

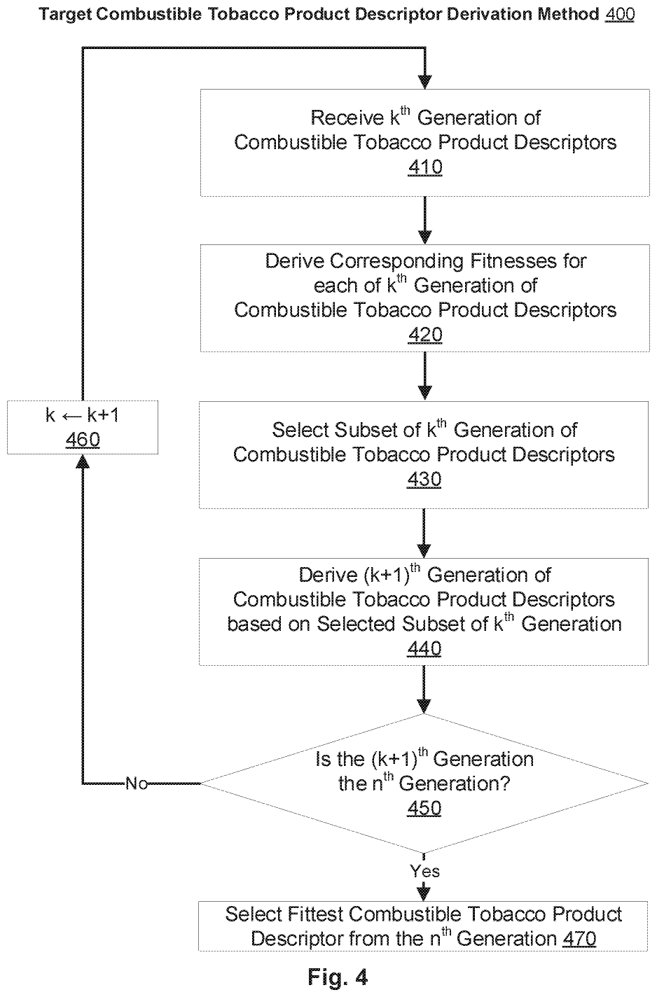

FIG. 2 is a schematic block diagram illustrating an example embodiment of the component 110 of the combustible tobacco product design system 100 for calculating design parameters for a combustible tobacco product. The illustrated example embodiment may perform the combustible tobacco product optimization method 400 described in relation to FIG. 4.

The illustrated embodiment of the combustible tobacco product design parameter calculator 110 includes a descriptor source 210, a descriptor fitness evaluator 220, a descriptor selector 230, a child descriptor generator 240, a descriptor mutator 250 and a descriptor receiver 260. The illustrated combustible tobacco product design parameter calculator uses these included components to perform one or more processing iterations in which combustible tobacco product descriptors are generated.

The descriptor source 210 is a source of combustible tobacco product descriptors. The descriptor source may be a source of stored combustible tobacco product descriptors 120. These stored combustible tobacco product descriptors 120 may be retrieved by the descriptor source 210 from a suitable data storage system, such as a database or file storage system, or from an in-memory cache. Where combustible tobacco product descriptors have already been generated, e.g. in a preceding iteration, the descriptor source may also be a source of these generated combustible tobacco product descriptors. These generated combustible tobacco product descriptors may have been retrieved or received from the descriptor receiver 260.

The descriptor fitness evaluator 220 receives combustible tobacco product descriptors from the descriptor source 210. The received combustible tobacco product descriptors may be a set of stored combustible tobacco product descriptors in the first iteration and, in subsequent iterations, may be the combustible tobacco product descriptors derived and/or otherwise received by the descriptor receiver 260 during the preceding iteration. The descriptor fitness evaluator evaluates the fitness of each of the received combustible tobacco product descriptors using a fitness function or loss function based on the input parameter values, as previously described.

The descriptor selector 230 receives the combustible tobacco product descriptors and associated fitness values from the descriptor fitness evaluator.

If the descriptor selector 230 determines that the final iteration has been reached then the descriptor selector may select the fittest combustible tobacco product descriptor of the received combustible tobacco product descriptors based on the associated fitness values and provide it to the descriptor receiver 260 with an indication that the final iteration has been reached. The descriptor selector 230 may determine that the final iteration has been reached if an iteration limit has been reached, e.g. the current iteration is the 100^{th} iteration and only a maximum of 100 iterations are to be performed. Alternatively, the descriptor selector 230 may determine that the final iteration has been reached if the fittest combustible tobacco product descriptor has a fitness greater than a threshold fitness, e.g. if the loss function is below a given value.

If the descriptor selector 230 does not determine that the final iteration has been reached, the descriptor selector may proceed with one or more of the following operations.

The descriptor selector 230 may select one or more elite descriptors and provide them to the descriptor receiver 260. The one or more elite descriptors may be the K combustible tobacco product descriptors of the received combustible tobacco product descriptors having the greatest fitnesses.

The descriptor selector may also select a plurality of parent combustible tobacco product descriptors and provide them to the child descriptor generator 240. The plurality of parent descriptors may be the N combustible tobacco product descriptors of the received combustible tobacco product descriptors having the greatest fitnesses, where N may be greater than K. Alternatively, a probabilistic procedure may be used, such as fitness proportionate selection, where the parent descriptors are selected by selecting descriptors from the received combustible tobacco product descriptors with a probability based on their fitness, i.e. combustible tobacco product descriptors with a greater fitness are more likely to be selected.

The descriptor selector 230 may also select one or more combustible tobacco product descriptors for mutation and provide them to the descriptor mutator 250. The one or more descriptors for mutation may be selected at random from the received combustible tobacco product descriptors or from a subset of the received combustible tobacco product descriptors, e.g. the fittest M of the received combustible tobacco product descriptors, or the parent tobacco product descriptors. The one or more descriptors for mutation may also be selected by selecting descriptors from the received combustible tobacco product descriptors with a probability based on their fitness.

The child descriptor generator 240 receives the plurality of parent combustible tobacco product descriptors from the descriptor selector and uses them to generate child combustible tobacco product descriptors. Each child combustible tobacco product descriptor may be generated by performing a crossover operation of two or more of the parents. The parents to be crossed over to generate each child may be chosen (pseudo)randomly or according to fixed combinations, e.g. the first parent with the second parent, the third parent with the fourth parent etc. The crossover operation may linearly combine two or more parent descriptors, with each of the parents weighted in the combination using a (pseudo)random variable. For example, where two parent descriptors, x and y, are used to generate a child descriptor, c, the child descriptor may be:

c=αx÷(1−α)y

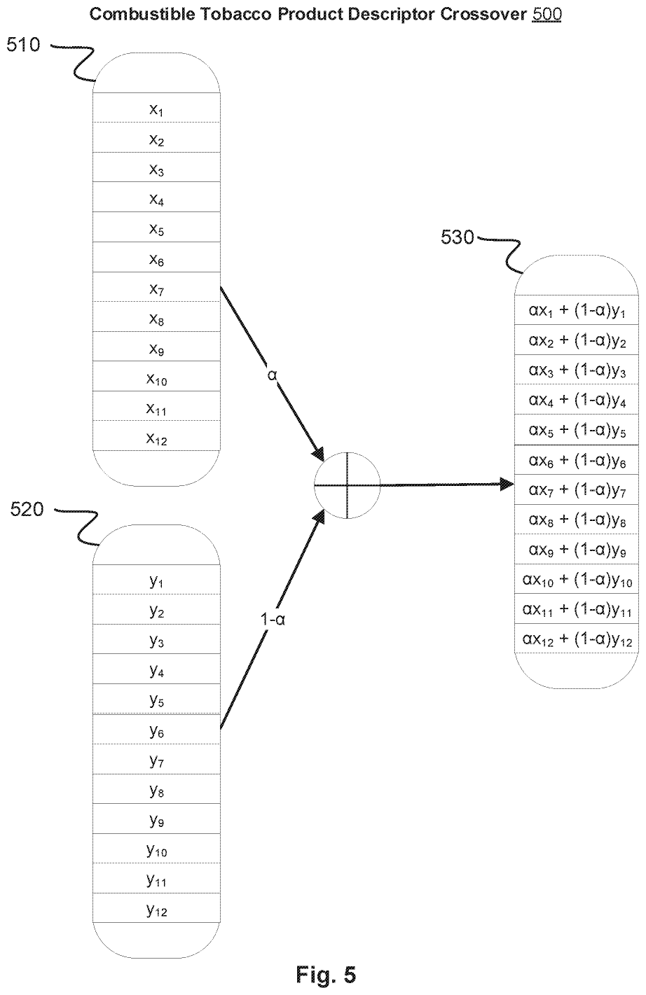

, where α is a (pseudo)random variable between 0 and 1, as illustrated in FIG. 5.

The descriptor mutator 250 may receive the one or more combustible tobacco product descriptors for mutation from the descriptor selector and uses them to generate mutated combustible tobacco product descriptors. Alternatively or additionally, the descriptor mutator may receive one or more child combustible tobacco product descriptors for mutation from the child descriptor generator. Each mutated combustible tobacco product descriptor may be generated by performing a crossover operation of a descriptor for mutation with a stored combustible tobacco product descriptor received via the descriptor source 210. The crossover operation may linearly combine a descriptor for mutation with a stored combustible tobacco product descriptor, with each weighted in the combination using a (pseudo)random variable. For example, where a descriptor for mutation, d, and a stored descriptor, s, are used to generate a mutated descriptor, m, the mutated descriptor may be:

m=(1−β)d+βs

, where β is a pseudo(random) variable between 0 and 1. β may be constrained to be or be more likely to be towards the lower end of this stated range, e.g. between 0 and 0.1.

If the descriptor receiver 260 receives an indication that the final iteration has been reached, the descriptor receiver 260 also receives the fittest combustible tobacco product descriptor of the final iteration, which is the target combustible tobacco product descriptor. The descriptor receiver 260 uses the target combustible tobacco product descriptor to obtain the design parameter values, as previously described, and provides them as an output.

Otherwise, the descriptor receiver 260 receives the one or more elite combustible tobacco product descriptors; the child combustible tobacco product descriptors; and the one or more mutated combustible tobacco product descriptors. The descriptor receiver may provide the combustible tobacco product descriptors which it has received to the descriptor source 210.

Combustible Tobacco Product Design Method

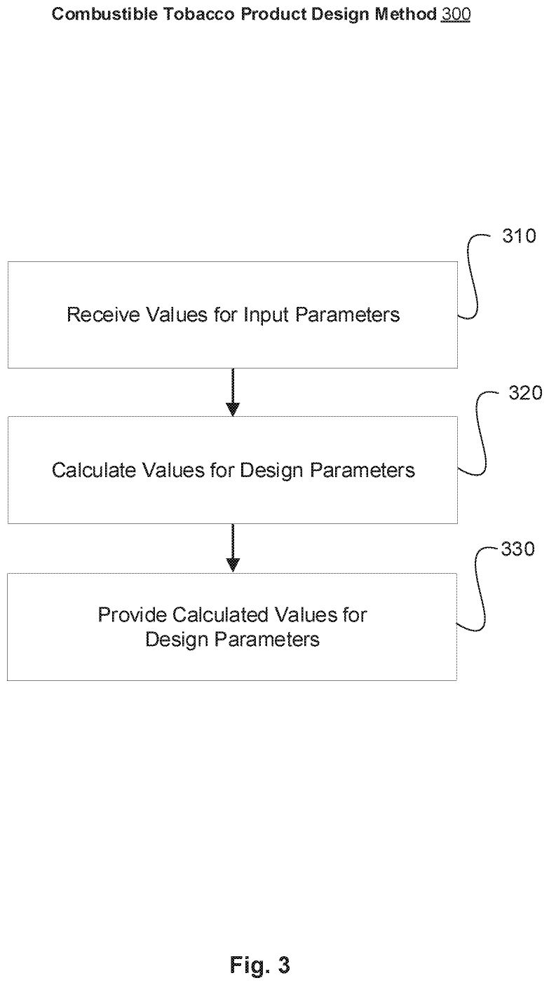

FIG. 3 is a flow diagram illustrating an example method for designing a target combustible tobacco product. The method may be performed by executing computer-readable instructions using one or more processors of one or more computing devices, e.g. the one or more computing devices implementing the combustible tobacco product design system 100.

In step 310, values for a plurality of input parameters are received. The values for the plurality of input parameters are desired and/or set values for parameters of the target combustible tobacco product. The parameters may include, but are not limited to, one or more of tobacco blend parameters; smoke sensory attributes; tar, nicotine and/or carbon monoxide deliveries; and parameters describing the physical properties and/or composition of a combustible tobacco product. Examples of such parameters are described in detail in relation to the input parameter values 101 of combustible tobacco product design system 100.

In step 320, values for a plurality of design parameters for the target combustible tobacco product are calculated based on the received values for the plurality of input parameters. The design parameters may be any number of the parameters described above as being usable as input parameters. The design parameters may include one or more parameters of the combustible tobacco product which were not input parameters.

The plurality of values for the design parameters may be calculated such that the target combustible tobacco product has the received values for the plurality of input parameters, or as close as is achievable. For example, the values for the plurality of input parameters may indicate that the target combustible tobacco product is desired to have certain sensory attribute values and have a blend consisting of given tobacco varieties; and the values for the design parameters may describe the physical properties and/or composition of the target combustible tobacco product and the proportions of the tobacco varieties in the blend such that the target combustible tobacco product has properties matching, or at least resembling, the received values for the input parameters.

The calculation of the values for the plurality of design parameters may include deriving a target combustible tobacco product descriptor. Combustible tobacco product descriptors may include values for the plurality of design parameters and values for the plurality of input parameters. These value of a given combustible tobacco product descriptor may be unscaled or may have undergone feature scaling, as described in relation to the deriving of combustible tobacco product descriptors in the example combustible tobacco product design system 100. Where the values of the target combustible tobacco product descriptor have undergone feature scaling, the calculation of the values for the plurality of design parameters may include transforming at least the values of the target combustible tobacco product descriptor for the plurality of design parameters into a scale appropriate for being provided as an output. For example, the values may be transformed into a scale understandable by a designer of combustible tobacco products and/or usable for manufacturing the target combustible tobacco product.

Combustible tobacco product descriptors may be implemented using any suitable data structure. Suitable data structures include, but are not limited to, arrays, vectors, matrices, rows and/or columns of matrices, in-memory objects, markup language files, serialized binary data, database entries and text data.

The target combustible tobacco product descriptor may be derived by performing an optimization procedure, which may be a stochastic optimization procedure. For example, the optimization procedure may be any of particle swarm optimization, ant colony optimization, simulated annealing, a Monte Carlo algorithm, Runge-Kutte methods, a genetic algorithm, or any combination thereof. Where a genetic algorithm is used, it may be a real coded genetic algorithm. The optimization procedure may be directed towards deriving a target combustible tobacco product having a maximal fitness. The fitness of a given combustible tobacco product descriptor may be based on differences between the values for the plurality of input parameters, or a feature scaling thereof, and the corresponding values of the target combustible tobacco product descriptor.

The fitness of a given combustible tobacco product descriptor may be measured using a fitness function or loss function. Where a fitness function is used, a greater value of the fitness function for the given combustible tobacco product descriptor indicates a greater fitness. Where a loss function is used, a lesser value of the loss function for the combustible tobacco product descriptor indicates a greater fitness. For example, the fitness of a combustible tobacco product descriptor may be inversely related to the root mean square deviation, also referred to as the root mean square error, between the values for the plurality of input parameters, or a feature scaling thereof, and the corresponding values of the target combustible tobacco product descriptor, and, this root mean square deviation used as a loss function. This root mean square deviation may be denoted as:

$$\frac{1}{N} \sqrt{{\sum }_{i = 1}^{N} {( {p}_{i} - {c}_{i} )}^{2}} ,$$

where N is the number of input parameters, pᵢ is the value for the ith of the plurality of input parameters, or a feature scaling thereof, and cᵢ is the value of a given combustible tobacco product descriptor for the ith of the plurality of input parameters.

The calculation of the values for the plurality of design parameters may be based on a plurality of stored combustible tobacco product descriptors. For example, the target combustible tobacco product descriptor may be derived using the plurality of stored combustible tobacco descriptors. The stored combustible tobacco product descriptors may be implemented using any suitable data structure for combustible tobacco product descriptors, include those previously referred to. The plurality of stored combustible tobacco product descriptors may be retrieved from any suitable data storage mechanism storing the plurality, or a greater plurality, of combustible tobacco product descriptors, e.g. the stored combustible tobacco product descriptors may be retrieved from file system storage, database storage or an in-memory cache.

The target combustible tobacco product descriptor may be derived by using a plurality of the stored combustible tobacco product descriptors, or a feature scaling thereof, as initial combustible tobacco product descriptors. The fitness of the initial combustible tobacco product descriptors may be evaluated and new combustible tobacco product descriptors may be derived based on a selected subset of them, e.g. the fittest J initial combustible tobacco product descriptors may be used to derive the new combustible tobacco product descriptors. The fitness of these new combustible tobacco product descriptors may then be evaluated and a selected subset of the new combustible tobacco product descriptors used to generate a further generation of combustible tobacco product descriptors. Subsequent generations may then be generated, each of the subsequent generations derived from a selected subset of the combustible tobacco product descriptors of the preceding generation. The target combustible tobacco product descriptor may be the fittest combustible tobacco product descriptor of the last generation. A related example method for deriving the target combustible product descriptor is described in relation to FIG. 4.

In operation 330, the values for the design parameters are provided as an output. The values for the design parameters may be displayed to a combustible tobacco product designer using a suitable graphical interface and/or may be used by a combustible tobacco product manufacturing apparatus to manufacture the target combustible tobacco product.

Combustible Tobacco Product Descriptor Optimization Method

FIG. 4 is a flow diagram illustrating an example method 400 for deriving a target combustible tobacco product descriptor. The method may be performed by executing computer-readable instructions using one or more processors of one or more computing devices, e.g. the one or more computing devices implementing the combustible tobacco product design system 100.

The described operations are repeated for a number of iterations. A total of (n−1) iterations are performed to derive an nth generation of combustible tobacco product descriptors. The number n is an integer greater than or equal to two. The number n may be a fixed number or may denote the generation in which an end criterion is met. For example, n may denote the generation in which the fittest combustible tobacco product descriptor has a fitness greater than a threshold fitness, e.g. the loss function value for that descriptor is below a given value.

In operation 410, the kth generation of combustible tobacco product descriptors is received. If the kth generation is the first generation of combustible tobacco product descriptors, the received combustible tobacco product descriptors may be received from a suitable data storage system, such as a database or file storage system, or from an in-memory cache. Otherwise, the received combustible tobacco product descriptors may be those derived in the preceding generation.

In operation 420, corresponding fitnesses for each of the kth generation of combustible tobacco product descriptors are derived. The fitness of each of the kth generation of combustible tobacco product descriptors may be derived using a fitness function or loss function based on the values of the respective combustible tobacco product descriptor for the input parameters, as previously described.

In operation 430, one or more subsets of the kth generation of combustible tobacco product descriptors are selected.

An elite subset of combustible tobacco product descriptors may be selected. The elite subset of combustible tobacco product descriptors may be the M combustible tobacco product descriptors of the kth generation of combustible tobacco product descriptors having the greatest fitnesses.

A parent subset of combustible tobacco product descriptors may be selected. The parent subset may be the M combustible tobacco product descriptors of the combustible tobacco product descriptors kth generation of combustible tobacco product descriptors having the greatest fitnesses, where M may be greater than K. Alternatively, a probabilistic procedure may be used to select the parent subset, such as fitness proportionate selection, where the parent descriptors are selected by selecting descriptors from the kth generation of combustible tobacco product descriptors with a probability based on their fitness, i.e. combustible tobacco product descriptors with a greater fitness are more likely to be selected.

A mutatee subset of combustible tobacco product descriptors may be selected. The mutatee subset may be selected at random from the kth generation of combustible tobacco product descriptors or from a subset of the kth generation of combustible tobacco product descriptors, e.g. the fittest M of the kth generation of combustible tobacco product descriptors, or the parent subset of kth generation of combustible tobacco product descriptors. The mutatee subset may also be selected by selecting descriptors from kth generation of combustible tobacco product descriptors with a probability based on their fitness.

In operation 440, a (k+1)th generation of combustible tobacco product descriptors is derived based on the one or more selected subsets of the kth generation of combustible tobacco product descriptors.

The (k+1)th generation of combustible tobacco product descriptors may include the elite subset of the kth generation of combustible tobacco product descriptors.

The (k+1)th generation of combustible tobacco product descriptors may include child descriptors derived based on the parent subset of the kth generation of combustible tobacco product descriptors. Each child combustible tobacco product descriptor may be generated by performing a crossover operation of two or more of the parent subset. The parents to be crossed over to generate each child may be chosen (pseudo)randomly or according to fixed combinations, e.g. the first parent with the second parent, the third parent with the fourth parent etc. The crossover operation may linearly combine two or more of the descriptors in the parent subset, with each of the parents weighted in the combination using a (pseudo)random variable. For example, where two parent descriptors, x and y, are used to generate a child descriptor, c, the child descriptor may be:

c=αx+(1−α)y

, where α is a (pseudo)random variable between 0 and 1, as illustrated in FIG. 5.

The (k+1)th generation of combustible tobacco product descriptors may include mutated combustible tobacco product descriptors derived based on the mutatee subset of the kth generation of combustible tobacco product descriptors. The (k+1)th generation of combustible tobacco product descriptors may also include mutated combustible tobacco product descriptors derived based on a mutatee subset of the child combustible tobacco product descriptors. Each mutated combustible tobacco product descriptor may be generated by performing a crossover operation of a descriptor from a mutatee subset with a stored combustible tobacco product descriptor. The crossover operation may linearly combine a combustible tobacco product descriptor from a mutatee subset with a stored combustible tobacco product descriptor, with each weighted in the combination using a (pseudo)random variable. For example, where a mutatee descriptor, d, and a stored descriptor, s, are used to generate a mutated descriptor, m, the mutated descriptor may be:

m=(1−β)d+βs

, where β is a pseudo(random) variable between 0 and 1. β may be constrained to be or be more likely to be towards the lower end of this stated range, e.g. between 0 and 0.1.

In operation 450, it is determined whether the (k+1)th generation of descriptors is the nth generation of descriptors. Where there are a fixed number of iterations are performed, the determination may comprise determining whether (k+1)is equal to n. In embodiments where n denotes that an end criterion is met, determining whether the (k+1)th generation is the nth generation includes the determining whether the (k+1)th generation of descriptors satisfies the end criterion. For example, it may be determined whether the fittest combustible tobacco product descriptor of the (k+1)th generation has a fitness greater than a threshold fitness, e.g. the loss function value for that descriptor is below a given value. In response to it being determined that the (k+1)th generation of descriptors is the nth generation of descriptors, the method continues to operation 470. Otherwise, the method continues to operation 460.

Operation 460 indicates that the operations described above are to be repeated for the next generation. The value k may be understood to have been incremented to (k+1). In some embodiments, a variable storing the value of or a value relating to k may be increment, e.g. embodiments using a for loop and a fixed number of iterations. However, in other embodiments, no such variable may be used or maintained and instead the illustrated incrementing of k merely denotes that execution continues for the next generation.

In operation 470, the combustible tobacco product descriptor of the nth generation of combustible tobacco product descriptors having the greatest fitness is selected as the target combustible tobacco product descriptor. As described in step 320 of method 300, the target combustible tobacco product descriptor is usable to derive values for the plurality of design parameters.

Combustible Tobacco Product Descriptor Crossover Example

FIG. 5 illustrates performing an example crossover operation 500 to derive a new combustible tobacco product descriptor based on existing combustible tobacco product descriptors. The described crossover operation may be performed by the child descriptor generator 240 and/or the descriptor mutator of the combustible tobacco product design parameter calculator 110 described in relation to FIG. 2. The described crossover operation may also be performed in child generation and/or mutation operations performed in the descriptor generation derivation operation 440 of the target combustible product optimization method 400.

The example crossover operation 500 includes a first combustible tobacco product descriptor 510, a second combustible tobacco product descriptor 520 and a derived combustible tobacco product descriptor 530.

The first combustible tobacco product descriptor 510 is a combustible tobacco product descriptor implemented as described above in relation to the system 100 and/or the method 300. The first combustible tobacco product descriptor 510 may be a stored combustible tobacco product descriptor; a combustible tobacco product descriptor derived in a preceding iteration of combustible tobacco product descriptor derivations; or a combustible tobacco product descriptor derived during the present iteration, e.g. a child combustible tobacco product descriptor which is to undergo mutation. The first combustible tobacco product descriptor 510 may be represented as a vector, x, having elements xᵢ. Each of the elements may be a value for a respective input or design parameter. In the illustrated example, the first combustible tobacco product descriptor 510 has 12 elements, x₁-x₁₂.

The second combustible tobacco product descriptor 520 is also a combustible tobacco product descriptor implemented as described above in relation to the system 100 and/or the method 300. The second combustible tobacco product descriptor 520 may be a stored combustible tobacco product descriptor; a combustible tobacco product descriptor derived in a preceding iteration of combustible tobacco product descriptor derivations; or a combustible tobacco product descriptor derived during the present iteration, e.g. a child combustible tobacco product descriptor which is to undergo mutation. The second combustible tobacco product descriptor 520 may be represented as a vector, y, having elements yᵢ. Each of the elements may be a value for a respective input or design parameter. Each of the elements, yᵢ, may be a value for the same respective input or design parameter as the corresponding element of the first combustible tobacco product descriptor, xᵢ. In the illustrated example, the second combustible tobacco product descriptor 520 has 12 elements, y₁-y₁₂, which are values for the same 12 parameters as those in the first combustible tobacco product descriptor, x₁-x₁₂,

The derived combustible tobacco product descriptor 530 is derived by linearly combining, e.g. calculating a weighted sum of, the first combustible tobacco product descriptor 510 and the second combustible tobacco product descriptor 520. In the illustrated example, the derived combustible tobacco product descriptor 530 is derived using a (pseudo)randomly generated number, a, which is in the range 0 to 1, and the derived combustible product descriptor is the sum of the first combustible tobacco product descriptor 510 multiplied by α and the second combustible product descriptor 520 multiplied by (1−α), i.e.:

z=αx+(1−α)y

, where z is a vector representing the derived combustible tobacco product descriptor 530. Therefore, as illustrated, the elements, zᵢ, of z are:

zᵢ=αxᵢ+(1−α)yᵢ

Combustible Tobacco Product

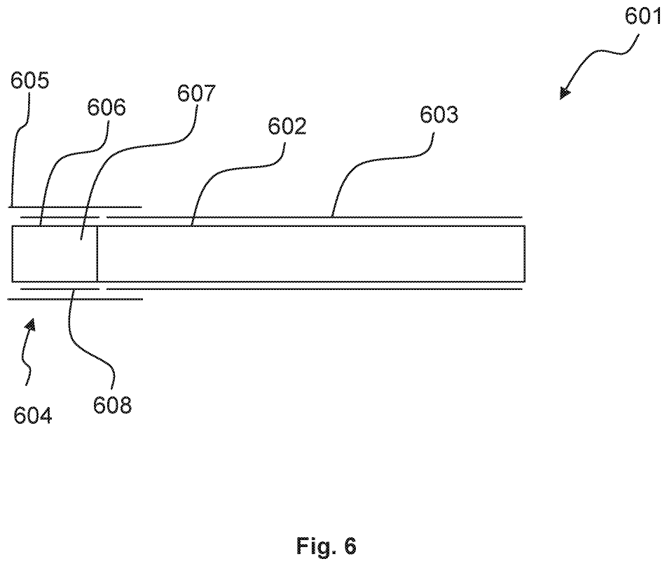

FIG. 6 is a schematic illustration of a filtered combustible tobacco product 601.

While the illustrated combustible tobacco product 601 is a filtered combustible tobacco product, the systems and methods described in the present specification are also applicable to unfiltered combustible tobacco products.

Filtered combustible tobacco products such as cigarettes and their formats are often named according to the cigarette length: “regular” (typically in the range of 68-75 mm, e.g. from about 68 mm to about 72 mm), “short” or “mini” (68 mm or less), “king-size” (typically in the range of 75-91 mm, e.g. from about 79 mm to about 88 mm), “long” or “super-king” (typically in the range of 91-105 mm, e.g. from about 94 mm to about 101 mm) and “ultra-long” (typically in the range from about 110 mm to about 121 mm).

They are also named according to the cigarette circumference: “regular” (about 23-25 mm), “wide” (greater than 25 mm), “slim” (about 22-23 mm), “demi-slim” (about 19-22 mm), “super-slim” (about 16-19 mm), and “micro-slim” (less than about 16 mm). Accordingly, a cigarette in a king-size, super-slim format will, for example, have a length of about 83 mm and a circumference of about 17 mm. Cigarettes in the regular, king-size format are preferred by many customers, namely with a circumference of from 23 to 25 mm and an overall length of from 75 to 91 mm.

Each format may be produced with filters of different lengths, smaller filters being generally used in formats of smaller lengths and circumferences. Typically the filter length will be from 15 mm, associated with short, regular formats, to 30 mm, associated with ultra-long super-slim formats. The tipping paper will have a greater length than the filter, for example from 3 to 10 mm longer.

The systems and methods described in the present specification are applicable to filtered combustible tobacco products in any of the above formats. The dimensions of a given filtered combustible tobacco product, whether actual, simulated or designed, in any of the above formats may be values of a combustible tobacco product descriptor for input parameters and/or design parameters.

The illustrated combustible tobacco product 601 is a smoking article that is generally cylindrical in shape and is in the regular, king size format, namely having a length in the range 75-91 mm and a circumference in the range 23 to 25 mm. The illustrated combustible tobacco product 601 may be an actual, simulated or designed combustible tobacco product and represented using a corresponding combustible tobacco product descriptor including values for a plurality of input parameters and design parameters. The length and circumference of the illustrated tobacco product 601, or a feature scaling thereof, may be values included in the corresponding combustible tobacco product descriptor. Values indicative of other properties of the cigarette, such as its firmness, density and the pressure drop through the cigarette may also be included in the corresponding combustible tobacco product descriptor, as previously described.

The illustrated combustible tobacco product 601 includes a tobacco rod 602. The tobacco rod 602 may include tobacco of a given tobacco blend composition. Values indicative of the given tobacco blend composition may be included in the corresponding combustible tobacco product descriptor. Values indicative of the weight and density of the tobacco rod 602 may also be included in the corresponding combustible tobacco product descriptor.

The tobacco rod is wrapped in a wrapping material 603, in this example cigarette paper, connected longitudinally to a filter 604 by tipping material 605 overlaying the filter 604 and partially overlaying the wrapping material 603 so as to connect the filter 604 to the tobacco rod 602. A value indicative of the lengths of the tipping material may be included in the corresponding combustible tobacco product descriptor. A value indicative of the porosity of the wrapping material may also be included in the corresponding combustible product descriptor. A value indicative of the burning additive (citrate) loading of the wrapping material may also be included in the corresponding combustible product descriptor.

The filter 604 includes a filter plug 606 formed using continuous cellulose acetate fibers and a plasticizer wrapped in a plug wrap 608. A value indicative of the length of the filter plug may be included in the corresponding combustible tobacco product descriptor. The filter plug includes absorbent material 607. The properties of the filter plug 606, including the properties of the absorbent material 607, may affect the pressure drop across the filter plug. A value indicative of the pressure drop across the filter plug may be included in the corresponding combustible tobacco product descriptor. Values indicative of the properties of the absorbent material 607 may also be included in the corresponding combustible tobacco product descriptor.

The combustible tobacco product 601 is, in the present example, provided with ventilation holes (not shown) through the tipping material 605 and plug wrap 608, providing ventilation into the filter plug 606. The ventilation holes may be described using a ventilation rate. A value indicative of the ventilation rate may be included in the corresponding combustible product descriptor.

In use, the tobacco rod 602 of the combustible tobacco product 601 is lit by a consumer in the conventional manner and tobacco smoke is drawn from burning coal of the tobacco rod 602 through the filter 604. The smoke sensory attributes of the combustible tobacco product may be assessed in use by consumers of the combustible tobacco product. Values indicative of the consumers' impressions of these smoke sensory attributes may be included in the corresponding combustible tobacco product descriptor.

Simulation Results Evaluations

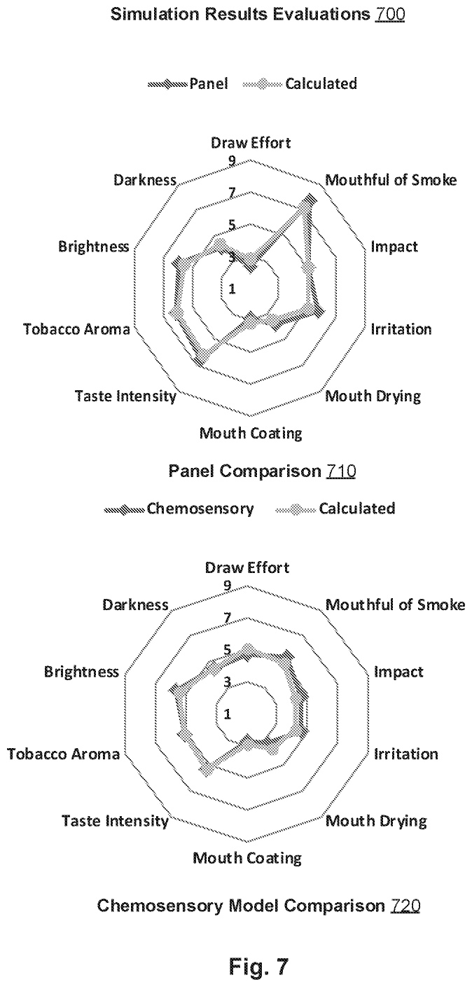

FIG. 7 is an illustration 700 of comparisons of estimates of smoke sensory attributes of a combustible tobacco product derived according to example embodiments with sensory attribute values obtained using other methods. Smoke sensory attributes may be estimated by example embodiments of the described systems and methods by using known properties of a combustible tobacco product as input parameters, e.g. blend composition parameters and physical properties of the cigarette, and the smoke sensory attributes as the design parameters.

The illustration 700 includes a panel comparison graph 710 and a chemosensory model comparison graph 720.

The panel comparison graph 710 compares results for smoke sensory attributes estimated by an embodiment of the method described herein with the results provided by a panel of consumers evaluating the smoke sensory attributes. As the graph 710 illustrates, the results estimated by the embodiment are close to those given by the panel of consumers. Therefore, the described systems and methods may reduce the number of consumer evaluations, e.g. using surveys or focus groups, undertaken to evaluate combustible tobacco products during the design process.

The chemosensory model comparison graph 720 compares results for smoke sensory attributes estimated by an embodiment of the method described herein with the results provided using a chemosensory model. As the graph 720 illustrates, the results estimated by the embodiment are close to those given by the chemosensory model. The chemosensory model uses chemical fingerprints to estimate the smoke sensory attributes. Chemical fingerprints are information dense and require a significant amount of processing. The chemosensory model uses more computational resources than the described systems and methods. Therefore, the described systems and method may reduce the computational resources used, e.g. using surveys or focus groups, to derive accurate estimates for the smoke sensory attributes of a combustible tobacco product.

In order to address various issues and advance the art, the entirety of this disclosure shows by way of illustration various embodiments in which the claimed invention(s) may be practiced and provide for superior design and simulation of combustible tobacco products. The advantages and features of the disclosure are of a representative sample of embodiments only, and are not exhaustive and/or exclusive. They are presented only to assist in understanding and teach the claimed features. It is to be understood that advantages, embodiments, examples, functions, features, structures, and/or other aspects of the disclosure are not to be considered limitations on the disclosure as defined by the claims or limitations on equivalents to the claims, and that other embodiments may be utilized and modifications may be made without departing from the scope and/or spirit of the disclosure. Various embodiments may suitably comprise, consist of, or consist essentially of, various combinations of the disclosed elements, components, features, parts, steps, means, etc. In addition, the disclosure includes other inventions not presently claimed, but which may be claimed in future.

1. A method of designing a target combustible tobacco product, the method comprising:
receiving respective values for a plurality of input parameters;
calculating respective values for a plurality of design parameters for the target combustible tobacco product based on the received values for the plurality of input parameters, the plurality of design parameters comprising at least two parameters selected from:
a tobacco blend composition;
tar, nicotine and carbon monoxide deliveries;
a smoke sensory attribute;
a number of puffs associated with the target combustible tobacco product;
combustible tobacco product dimensions;
tobacco weight;
tobacco rod and/or filter density;
tobacco rod and/or filter firmness;
open and/or closed combustible tobacco product pressure drop;
filter pressure drop;
cigarette paper porosity; and
ventilation level; and
providing the calculated values as an output.

2. The method of claim 1, wherein the calculation of the values for the design parameters comprises deriving a target combustible tobacco product descriptor, wherein the target combustible tobacco product descriptor comprises values for the design parameters and values for the input parameters for the target combustible tobacco product.

3. The method of claim 2, wherein the deriving a target combustible tobacco product descriptor comprises performing an optimization procedure directed to deriving a target combustible tobacco product descriptor having a maximal fitness.

4. The method of claim 3, wherein the fitness of a given combustible tobacco product descriptor is based on differences between the values of the given combustible tobacco product descriptor for the input parameters and corresponding values based on the received values for the input parameters.

5. The method of claim 4, wherein the fitness of a given combustible tobacco product is inversely related to the root mean square deviation between the values of the given combustible product descriptor for the input parameters and corresponding values based on the received values for the input parameters.

6. The method of claim 3, wherein the performing of the optimization procedure comprises, repeating for each k between 1 and (n−1), where n≥2:
receiving a kth generation of combustible tobacco product descriptors;
deriving corresponding fitnesses for each of the kth generation of combustible tobacco product descriptors;
selecting one or more subsets of the kth generation of combustible tobacco product descriptors based on the corresponding fitnesses; and
deriving a (k+1)th generation of combustible tobacco product descriptors based on the one or more subsets of the kth generation of combustible tobacco product descriptors,
wherein the target combustible product descriptor is the combustible tobacco product descriptor of the nth generation having the greatest fitness.

7. The method of claim 6, wherein deriving the (k+1)th generation of combustible tobacco product descriptors comprises deriving one or more child combustible tobacco product descriptors, wherein each of the one or more child combustible tobacco product descriptors is based on a respective two or more of the subset of the kth generation of combustible tobacco product descriptors.

8. The method of claim 7, wherein each of the one or more child combustible tobacco product descriptors is a linear combination of the respective two or more of the subset of the kth generation of combustible tobacco product descriptors.

9. The method of claim 7, wherein deriving the (k+1)th generation of combustible tobacco product descriptors comprises mutating at least one of the one or more child combustible tobacco product descriptors.

10. The method claim 3, wherein the optimization procedure is a stochastic optimization procedure.

11. The method of claim 10, wherein the stochastic optimization procedure is a genetic algorithm.

12. The method of claim 11, wherein the genetic algorithm is a real coded genetic algorithm.

13. The method of claim 10, wherein the optimization procedure comprises at least one selected from particle swarm optimization, ant colony optimization, simulated annealing, a Monte Carlo algorithm, Runge-Kutte methods, a genetic algorithm, or any combination thereof.

14. The method of claim 1, wherein the values for the plurality of design parameters are calculated based on a plurality of stored combustible tobacco product descriptors, wherein each of the stored combustible tobacco product descriptors comprises values for the plurality of design parameters and values for the plurality of input parameters for a corresponding combustible tobacco product.

15. The method of claim 14, further comprising deriving one or more of the plurality of stored combustible tobacco product descriptors using chemometric analysis.

16. The method of claim 1, wherein the plurality of input parameters comprise at least two parameters selected from:
a tobacco blend composition;
tar, nicotine and carbon monoxide deliveries;
a smoke sensory attribute;
a number of puffs associated with the target combustible tobacco product;
combustible tobacco product dimensions;
tobacco weight;
tobacco rod and/or filter density;
tobacco rod and/or filter firmness;
open and/or closed combustible tobacco product pressure drop;
filter pressure drop;
cigarette paper porosity; and
ventilation level.

17. The method claim 1, further comprising manufacturing the target combustible tobacco product based on the calculated values for the plurality of design parameters.

18. A computer program comprising instructions which, when the program is executed by a computer, cause the computer to carry out the method of claim 1.

19. A computer-readable storage medium comprising instructions which, when executed by a computer, cause the computer to carry out the method claim 1.

20. A data processing apparatus comprising a processor and a computer-readable storage medium as claimed in claim 19.

21. A system comprising:
a data processing apparatus comprising a computer and a computer-readable storage medium comprising instructions, which when executed by a computer, cause the computer to carry out the method of claim 1; and
a combustible tobacco product manufacturing apparatus configured to manufacture the target combustible tobacco product based on the calculated values for the plurality of design parameters.
